(19) United States
(12) Patent Application Publication
(10) Pub. No.: US 2013/0084260 A1
(43) Pub. Date: Apr. 4, 2013

(54) HAIR CARE COMPOSITION

(71) Applicants: Samiul Amin, Bromborough (GB); Prem Kumar Cheyalazhagan Paul, Bebington (GB); Pamela Eileen Red, Bebington (GB)

(72) Inventors: Samiul Amin, Bromborough (GB); Prem Kumar Cheyalazhagan Paul, Bebington (GB); Pamela Eileen Red, Bebington (GB)

(21) Appl. No.: 13/700,747

(22) PCT Filed: Jun. 2, 2011
(86) PCT No.: PCT/EP11/59171

§ 371 Date: Nov. 29, 2012

(30) Foreign Application Priority Data
Jun. 10, 2010 (EP) 10165540.5

(51) Int. Cl. A61K 8/26; A61K 8/28; A61Q 5/12; A61K 8/891; A61K 8/06; A61K 8/892; A61K 8/19; A61K 8/20

(52) U.S. Cl. CPC A61K 8/26; A61K 8/19; A61K 8/28; A61K 8/20; A61K 8/891; A61K 8/06; A61K 8/892; A61Q 5/12
USPC 424/70.1

(57) ABSTRACT

A method of treating hair comprising the following steps: i) applying to the hair a composition comprising a group III or group IV metal salt or combination thereof; ii) followed by washing the hair.

### FIELD OF THE INVENTION

The present invention relates to a pre-treatment composition for hair preventing the hair from tangling.

### BACKGROUND

Compositions that can be used to condition hair are well known. Hair conditioning compositions are intended to leave the hair manageable, soft, shiny and easy to comb. Most hair conditioning compositions are applied to the hair when wet, usually as an after-treatment following shampooing. More recently, two-in-one conditioning shampoos have been developed which provide cleansing and conditioning of the hair with a single composition.

These conditioning shampoos and conditioners help in detangling the hair. However such compositions can leave the hair feeing greasy and heavy.

There remains a need for a product that prevents tangle formation and leaves the hair without the sensory negatives described above.

### DESCRIPTION

The present invention relates to a method of treating hair comprising the following steps:
i) applying to the hair a composition comprising a group III or group IV metal salt or combination thereof;
ii) followed within 30 minutes of applying the composition by washing the hair with a surfactant containing shampoo.

### DETAILED DESCRIPTION

### Metal Salts

Compositions of the invention comprise a group III or group IV metal salt the metal salt, preferably the metal salt is an aluminium or zirconium salt.

Chloride salts are advantageous. It is particularly preferred if the metal salt is aluminium chloride.

The level of metal salt is preferably from 0.01 to 10 wt % of the total composition, more preferably from 0.2 to 5 wt %.

### Silicones

Compositions of the invention preferably comprise a silicone, more preferably in the form of an emulsion.

Amino-functional polydimethyl siloxanes which have the CTFA designation amodimethicone may be used with the present invention. Preferred silicones are emulsions and include silicones such as polydiorganosiloxanes, in particular polydimethylsiloxanes which have the CTFA designation dimethicone, polydimethyl siloxanes having hydroxyl end groups which have the CTFA designation dimethiconol.

The emulsion droplets may typically have a Sauter mean droplet diameter (D_{3,2}) in the composition of the invention ranging from 0.01 to 20 micrometer, more preferably from 0.2 to 10 micrometer.

A suitable method for measuring the Sauter mean droplet diameter (D_{3,2}) is by laser light scattering using an instrument such as a Malvern Mastersizer. Suitable silicone emulsions for use in compositions of the invention are available from suppliers of silicones such as Dow Corning and GE Silicones. The use of such pre-formed silicone emulsions is preferred for ease of processing and control of silicone particle size. Such pre-formed silicone emulsions will typically additionally comprise a suitable emulsifier such as an anionic or nonionic emulsifier, or mixture thereof, and may be prepared by a chemical emulsification process such as emulsion polymerisation, or by mechanical emulsification using a high shear mixer. Pre-formed silicone emulsions having a Sauter mean droplet diameter (D_{3,2}) of less than 0.15 micrometers are generally termed microemulsions.

Examples of suitable pre-formed silicone emulsions include emulsions DC2-1766, DC2-1784, DC-1785, DC-1786, DC-1788 and microemulsions DC2-1865 and DC2-1870, all available from Dow Corning. These are all emulsions/microemulsions of dimethiconol.

Suitable amodimethicone emulsions are DC939, DC7134 (from Dow Corning) and SME253 (from GE Silicones).

Silicone will generally be present in a composition of the invention at levels of from 0.05 to 20%, preferably 1 to 15%, more preferably from 2 to 10% by total weight of silicone based on the total weight of the composition.

Compositions of the invention are generally aqueous, i.e. they have water or an aqueous solution or a lyotropic liquid crystalline phase as their major component. Suitably, the composition will comprise from 50 to 98%, preferably from 60 to 90% water by weight based on the total weight of the composition.

### Product Form

Compositions of the invention are pre-treatment compositions in that they are applied to the hair. The hair is preferably dry before the pre-treatment composition is applied. Shortly after application (within 1 hour, more preferably within 5 minutes) the hair is washed then rinsed.

Hair is washed in the conventional manner using any commercially available shampoo.

A particularly preferred product for the pre-treatment product is a spray, mousse, gel, cream or lotion. Spray products are particularly preferred.

The Examples will now be illustrated with reference to the following non-limiting Examples. Inventions according to the invention are demonstrated by a number, comparative inventions are demonstrated by a letter.

### EXAMPLES

The following examples were prepared:

| | | Wt/% | | | |
|---|---|---|---|---|---|
| | | Example | Example | Example | Example |
| Ingredient | Chemical Name | A | B | 1 | 2 |
| DC1788 | Dimethiconol/ | 7.0 | | 7.0 | |
| | TEAS-DOBS | | | | |
| DC200 50 | Dimethicone | 6.0 | 6.0 | 6.0 | 6.0 |
| CST | | | | | |
| DC7134 | Dimethicone with | | 9.8 | | 9.8 |
| | Amodimethicone | | | | |
| Aluminium | Aluminium | | | 2.0 | 2.0 |
| chloride | Chloride | | | | |
| hexahydrate | | | | | |

| | |
|---|---|
| Water and | To 100 wt % |
| minors | |

The pre-treatment composition was applied to dry European hair switches. Hair was washed using a commercial shampoo, rinsed and dried. The level of tangling was measured by dragging the switch through a static comb on a mechanical testing machine (Instron) whilst recording force and distance. The total detangling energy is obtained by calculating the cumulative energy needed for detangling.

The following table shows the detangling energy in Joules.

| Example | Example Dosage | Detangling energy |
|---|---|---|
| A | 0.1 | 6.5 |
| 1 | 0.1 | 0.86 |
| B | 0.1 | 1.9 |
| 2 | 0.1 | 0.5 |
| water | 0.1 | 14.8 |
| 2 wt % aluminium chloride | 0.1 | 3.0 |
| hexahydrate aqeous solution | | |

The above results show the advantage of using aluminium chloride in a pre-treatment composition. It should be noted that silicone further enhances the effect. The non-amino silicone is more effective than the amino silicone in preventing tangling.

1. A method of treating hair comprising the following steps:
i) applying to the hair a composition comprising a group III or group IV metal salt or combination thereof;
ii) followed within 30 minutes of applying the composition by washing the hair with a surfactant containing shampoo.

2. A method according to claim 1 in which the metal salt of the composition is an aluminium or zirconium salt.

3. A method according to claim 1 in which the metal salt of the composition is a chloride.

4. A method according to claim 1 in which the metal salt is aluminium chloride.

5. A method according to claim 1 in which the level of metal salt within the composition is from 0.2 to 5 wt % of the total composition.

6. A method according to claim 1 in which the composition further comprises a silicone.

7. A method according to claim 6 in which the silicone is in the form of an silicone emulsion.)

8. A method according to claim 6 in which the silicone does not comprise an amino group.
